(19) United States
(12) Patent Application Publication
(10) Pub. No.: US 2025/0249086 A1
(43) Pub. Date: Aug. 7, 2025

(54) Protein-Saccharide Conjugation with Sodium Cyanoborohydride

(71) Applicant: Sanofi Pasteur Inc., Swiftwater, PA (US)

(72) Inventors: Maryalice Ginley, Cambridge, MA (US); Steven Hauser, Easton, PA (US); Christophe Neyra, Cambridge, MA (US)

(73) Assignee: Sanofi Pasteur Inc., Swiftwater, PA (US)

(21) Appl. No.: 18/854,372

(22) PCT Filed: Apr. 10, 2023
(86) PCT No.: PCT/US2023/018016

§ 371 Date: Oct. 4, 2024

Related U.S. Application Data
(60) Provisional application No. 63/329,547, filed on Apr. 11, 2022.

(30) Foreign Application Priority Data
May 10, 2022 (EP) 22172626.8

(51) Int. Cl. A61K 39/095; A61K 39/00; A61K 39/09; A61K 39/102; A61K 39/385; A61K 47/64; G01N 24/08

(52) U.S. Cl. CPC A61K 39/095; A61K 39/092; A61K 39/102; A61K 39/385; A61K 47/646; G01N 24/08; A61K 2039/54; A61K 2039/6037; A61K 2039/6068

(57) ABSTRACT

Methods and uses of conjugating saccharides to protein carriers are disclosed herein. Exemplary conjugates prepared according to those methods and uses are also disclosed. Additionally, methods for quantifying the amount of sodium borohydride in a sodium cyanoborohydride reagent are disclosed herein. Vaccine compositions as well as related methods and uses are also disclosed herein.

### CROSS-REFERENCE TO RELATED APPLICATIONS

This application claims priority to U.S. Provisional Application No. 63/329,547, filed on Apr. 11, 2022, and European Patent Application No. 22172626.8 filed May 10, 2022, both of which are hereby incorporated by reference in its entirety.

### INTRODUCTION AND SUMMARY

Sodium cyanoborohydride (NaCNBH₃) is used in a variety of chemical reactions. For example, sodium cyanoborohydride reagents are often used in the conjugation of protein carriers to saccharides. Because sodium borohydride (NaBH₄) is a stronger reducing agent than sodium cyanoborohydride, the presence of sodium borohydride in a reaction has the potential to lead to impurities in the final product.

It was, however, unclear how much sodium borohydride in a sodium cyanoborohydride reagent would potentially interfere with the conjugation of protein carriers to saccharides and existing approaches for detecting sodium borohydride did not provide quantification of the amount of sodium borohydride in a sample of sodium cyanoborohydride and present additional challenges. For example, ¹¹B NMR is less commonly available than proton NMR (a.k.a., ¹H NMR) as ¹¹B NMR requires a tunable broadband probe. Further, typical NMR tubes, and sometimes the NMR probe itself, that are used for analysis are made of borosilicate glass. This type of glass, however, gives a broad background signal in the ¹¹B NMR spectrum that interferes with the baseline of the NMR spectrum, which can frustrate precise integration and quantitation.

It is described herein that even a small amount of sodium borohydride in sodium cyanoborohydride can adversely affect the conjugation reaction of a saccharide and protein carrier. Accordingly, this disclosure provides methods for conjugating at least one activated saccharide to at least one protein carrier, the method comprising reacting the at least one activated saccharide with the at least one protein carrier in the presence of a sodium cyanoborohydride reagent containing no more than about 0.7% of sodium borohydride to obtain a conjugate. Furthermore, it is reported that ¹H NMR is able to distinguish between sodium cyanoborohydride and sodium borohydride within a sample and can be used for relative quantification of sodium borohydride contained in samples of sodium cyanoborohydride.

In accordance with the description, methods and uses of conjugating saccharides to protein carriers are disclosed herein. Exemplary conjugates prepared according to those methods and uses are also disclosed herein. Additionally, methods for quantifying the amount of sodium borohydride in a sodium cyanoborohydride reagent are disclosed herein. Moreover, vaccine compositions as well as related methods and uses are also disclosed herein.

Accordingly, the following embodiments are provided. Embodiment 1 is a method for conjugating at least one activated saccharide to at least one protein carrier, the method comprising: (a) determining the amount of sodium borohydride in a sodium cyanoborohydride reagent; and (b) reacting the at least one activated saccharide with the at least one protein carrier in the presence of a sodium cyanoborohydride reagent containing no more than about 0.7% of sodium borohydride to obtain a conjugate.

Embodiment 2 is a use of a sodium cyanoborohydride reagent containing no more than about 0.7% of sodium borohydride as a reagent in conjugating at least one activated saccharide to at least one protein carrier by reductive amination, wherein the sodium cyanoborohydride reagent has been determined to contain no more than 0.7% sodium borohydride.

Embodiment 3 is use of a sodium cyanoborohydride reagent containing no more than about 0.7% of sodium borohydride as a reagent in conjugating at least one activated saccharide to at least one protein carrier by reductive amination, the use comprising: (a) determining the amount of sodium borohydride in a sodium cyanoborohydride reagent; and (b) reacting the at least one activated saccharide with the at least one protein carrier in the presence of a sodium cyanoborohydride reagent containing no more than about 0.7% of sodium borohydride to obtain a conjugate.

Embodiment 4 is a method or use according to any one of the preceding embodiments, wherein the method further comprises activating at least one saccharide with an activating agent to obtain the at least one activated saccharide.

Embodiment 5 is the method or use according to any one of the preceding embodiments, wherein the sodium cyanoborohydride reagent contains no more than about 0.6% of sodium borohydride.

Embodiment 6 is the method or use according to the immediately preceding embodiment, wherein the sodium cyanoborohydride reagent contains no more than about 0.5% of sodium borohydride.

Embodiment 7 is the method or use according to any one of the preceding embodiments, wherein the at least one activated saccharide comprises an activated form of a surface carbohydrate of a cell or virus.

Embodiment 8 is the method or use according to any one of the preceding embodiments, wherein the at least one activated saccharide comprises an activated form of a bacterial capsular polysaccharide.

Embodiment 9 is the method or use according to any one of the preceding embodiments, wherein the at least one activated saccharide comprises an activated form of a bacterial capsular polysaccharide from Haemophilus influenzae.

Embodiment 10 is the method or use according to any one of embodiments 1 to 8, wherein the at least one activated saccharide comprises an activated form of a bacterial capsular polysaccharide from Streptococcus pneumoniae.

Embodiment 11 is the method or use according to any one of embodiments 1 to 8, wherein the at least one activated saccharide comprises an activated form of a bacterial capsular polysaccharide from Neisseria meningitidis.

Embodiment 12 is the method or use according to the immediately preceding embodiment, wherein the Neisseria meningitidis is serogroup C, A, W-135 or Y

Embodiment 13 is the method or use according to any one of the preceding embodiments, wherein the at least one protein carrier comprises recombinant exoprotein alpha (REP A), Outer Membrane Protein Complex (OMPC), diphtheria toxoid, CRM197, tetanus toxoid or tetanus toxin C fragment.

Embodiment 14 is the method or use according to any one of the preceding embodiments, wherein the at least one protein carrier comprises diphtheria toxoid, CRM197 or tetanus toxoid.

Embodiment 15 is the method or use according to any one of the preceding embodiments, wherein the at least one protein carrier comprises tetanus toxoid.

Embodiment 16 is the method or use according to any one of the preceding embodiments, wherein the pH of the reaction is in a range from about 7 to about 10.

Embodiment 17 is the method or use according to any one of the preceding claims, wherein the pH of the reaction is about 8.

Embodiment 18 is the method or use according to any one of embodiments 1-16, wherein the pH of the reaction is about 9.

Embodiment 19 is the method or use of any one of the immediately preceding embodiments, wherein the determining the amount of sodium borohydride comprises performing NMR on the sodium cyanoborohydride reagent.

Embodiment 20 is the method or use of the immediately preceding embodiment, wherein the NMR comprises 1-dimensional proton NMR.

Embodiment 21 is a method for quantitating the amount of sodium borohydride in a sodium cyanoborohydride reagent, the method comprising: (a) subjecting a sample of a sodium cyanoborohydride reagent to 1-dimensional proton NMR, thereby obtaining NMR data; and (b) determining the amount of sodium borohydride in the sample from the NMR data.

Embodiment 22 is the method or use according to any one of embodiments 19-21, wherein the NMR is carried out in a deuterated solvent comprising the deuterated form(s) of at least one of dimethyl sulfoxide, chloroform, or methylene chloride.

Embodiment 23 is the method or use according to embodiments 19-22, wherein the NMR comprises at least one scan with a relaxation delay at about 41 to 50 seconds.

Embodiment 24 is the method or use according to embodiment 23, wherein the NMR comprises at least one scan with a relaxation delay at about 43 to 48 seconds.

Embodiment 25 is the method or use according to embodiment 24, wherein the NMR comprises at least one scan with a relaxation delay at about 45 seconds.

Embodiment 26 is the method or use according to any one of embodiments 19-25, wherein the NMR is carried out at a temperature ranging from about 20° C. to about 45° C.

Embodiment 27 is the method or use according to embodiment 26, wherein the NMR is carried out at a temperature ranging from about 28° C. to about 40° C.

Embodiment 28 is the method or use according to embodiment 27, wherein the NMR is carried out at a temperature ranging from about 30° C. to about 35° C.

Embodiment 29 is the method or use according to embodiment 28, wherein the NMR is carried out at a temperature of about 30° C.

Embodiment 30 is the method or use according to any one of embodiments 19-29, wherein the NMR is carried out with the parameters-temperature: about 30° C.; pulsewidth: pw90; spectral width: 7000 Hz; relaxation delay: about 45 second.

Embodiment 31 is the method or use according to any one of embodiments 19-30, wherein determining the amount of sodium borohydride comprises using the peak area at about 0.57 ppm for sodium borohydride resonance as referenced from a solvent resonance.

Embodiment 32 is the method or use of embodiment 31, wherein the solvent resonance comprises a dimethyl sulfoxide resonance at about 2.5 ppm.

Embodiment 33 is the method or use according to any one of embodiments 19-32, wherein determining the amount of sodium borohydride uses an external standard curve of sodium borohydride, optionally wherein the external standard curve of sodium borohydride is from about 20-120 μg/ml.

Embodiment 34 is the method or use according to embodiment 33, wherein the external standard curve comprises a 6-point external standard curve.

Embodiment 35 is a conjugate produced according to the method or use according to any one of embodiments 1-20 and 22-34.

Embodiment 36 is a vaccine composition comprising at least one protein conjugated saccharide obtained according to any one of embodiments 1-20 and 22-34.

Embodiment 37 is the vaccine composition of embodiment 36, further comprising a pharmaceutically acceptable buffer.

Embodiment 38 is the vaccine composition of embodiments 36 or 37, further comprising a pharmaceutically acceptable salt.

Embodiment 39 is the vaccine composition of any one of embodiments 36-38, which is formulated for intramuscular administration.

Embodiment 40 is a method of vaccinating a subject comprising administering a dose of the vaccine composition of any one of embodiments 36-39.

Embodiment 41 is a use of the vaccine composition of any one of embodiments 36-39 to immunize a subject.

Embodiment 42 is a use of the vaccine composition of any one of embodiments 36-39 for the manufacture of a medicament for immunizing a subject.

Embodiment 43 is the method or use according to any one of embodiments 40-42, wherein the subject is being immunized against Haemophilus influenzae.

Embodiment 44 is the method or use according to any one of embodiments 40-42, wherein the subject is being immunized against Neisseria meningitidis.

Embodiment 45 is the method or use according to any one of embodiments 40-42, wherein the subject is being immunized against Streptococcus pneumoniae.

Embodiment 46 is the method or use of any one of embodiments 40-45, wherein the vaccine composition is administered intramuscularly.

Embodiment 47 is a method of preparing a vaccine composition, comprising conjugating at least one activated saccharide to at least one protein carrier according to the method or use of any one of embodiments 1-20 or 22-34; and formulating the conjugate into a vaccine composition.

Embodiment 48 is a method of preparing at least one vaccine composition comprising a conjugate, the method comprising the steps of: (a) determining the amount of sodium borohydride in a sodium cyanoborohydride reagent, (b) selecting a sodium cyanoborohydride reagent containing no more than about 0.7% of sodium borohydride, (c) reacting at least one activated saccharide with at least one protein carrier in presence of the selected sodium cyanoborohydride reagent, thereby providing at least one conjugate, and (d) formulating the at least one conjugate into a vaccine composition.

Embodiment 49 is the method according to embodiment 48, wherein the method further comprises activating at least one saccharide with an activating agent to obtain the at least one activated saccharide.

Embodiment 50 is the method according to embodiments 48 or 49, wherein the selected sodium cyanoborohydride reagent contains no more than about 0.6% of sodium borohydride.

Embodiment 51 is the method according to the immediately preceding embodiment, wherein the selected sodium cyanoborohydride reagent contains no more than about 0.5% of sodium borohydride.

Embodiment 52 is the method according to any one of embodiments 48-51, wherein the at least one activated saccharide comprises an activated form of a surface carbohydrate of a cell or virus.

Embodiment 53 is the method according to any one of embodiments 48-52, wherein the at least one activated saccharide comprises an activated form of a bacterial capsular polysaccharide.

Embodiment 54 is the method according to any one of embodiments 48-53, wherein the at least one activated saccharide comprises an activated form of a bacterial capsular polysaccharide from Haemophilus influenzae.

Embodiment 55 is the method according to any one of embodiments 48-53, wherein the at least one activated saccharide comprises an activated form of a bacterial capsular polysaccharide from Streptococcus pneumoniae.

Embodiment 56 is the method according to any one of embodiments 48-53, wherein the at least one activated saccharide comprises an activated form of a bacterial capsular polysaccharide from Neisseria meningitidis.

Embodiment 57 is the method according to the immediately preceding embodiment, wherein the Neisseria meningitidis is serogroup C, A, W-135 or Y.

Embodiment 58 is the method according to any one of embodiments 48-57, wherein the at least one protein carrier comprises recombinant exoprotein alpha (REP A), Outer Membrane Protein Complex (OMPC), diphtheria toxoid, CRM197, tetanus toxoid or tetanus toxin C fragment.

Embodiment 59 is the method according to the immediately preceding embodiment, wherein the at least one protein carrier comprises diphtheria toxoid, CRM197 or tetanus toxoid.

Embodiment 60 is the method according to the immediately preceding embodiment, wherein the at least one protein carrier comprises tetanus toxoid.

Embodiment 61 is a conjugate for use as a vaccine according to any one of embodiments 36-39.

Embodiment 62 is a vaccine according to any one of embodiments 36-39 for use in preventing Haemophilus influenzae, Neisseria meningitidis or Streptococcus pneumoniae infection or disease.

Embodiment 63 is a vaccine according to any one of embodiments 36-39 for use in immunizing against Haemophilus influenzae, Neisseria meningitidis or Streptococcus pneumoniae.

Embodiment 64 is a conjugate for use in the manufacture of a vaccine according to any one of embodiments 36-39.

It is to be understood that both the foregoing general description and the following detailed description are exemplary and explanatory only and are not restrictive of the claims.

### BRIEF DESCRIPTION OF THE DRAWINGS

FIG. 1 shows the 1-D proton NMR spectrum of sodium borohydride and sodium cyanoborohydride as discussed in Example 1.

FIG. 2A shows the results of a temperature study on 1-D proton NMR spectra of sodium borohydride and sodium cyanoborohydride as discussed in Example 1.

FIG. 2B shows an overlay of peaks corresponding to borohydride from the NMR data shown in FIG. 2A.

FIG. 3 shows the results of a 1-D proton NMR study on various sodium cyanoborohydride samples as discussed in Example 1.

FIG. 4 shows integral values for sodium borohydride peaks as discussed in Example 1.

FIG. 5 shows an array of spectra from a sample of sodium cyanoborohydride spiked with sodium borohydride as discussed in Example 1.

FIG. 6 shows a linear curve generated using sodium borohydride concentration versus the peak area obtained from a sample of sodium borohydride in d-DMSO as discussed in Example 1.

FIG. 7 shows a linear curve generated using sodium borohydride concentration versus the peak area obtained from a sample of sodium cyanoborohydride spiked with variable amounts of sodium borohydride as discussed in Example 1.

FIG. 8 shows the linearity of the target (sodium borohydride) versus the observed sodium borohydride in a sample of sodium cyanoborohydride as discussed in Example 2.

FIG. 9 shows the HPSEC chromatograms (absorbance 280 nm) of a conjugation reaction between activated intermediate serogroup W-135 polysaccharide and tetanus toxoid as discussed in Example 3.

FIG. 10 shows the HPSEC chromatograms (absorbance 280 nm) of a conjugation reaction between activated intermediate serogroup Y polysaccharide and tetanus toxoid as discussed in Example 3.

FIG. 11 shows an enlargement of a region of the conjugate chromatogram shown in FIG. 10.

FIG. 12 shows the HPSEC chromatograms (absorbance 280 nm) of a conjugation reaction between intermediate serogroup C polysaccharide and tetanus toxoid as discussed in Example 3.

FIG. 13 shows the HPSEC chromatograms (absorbance 280 nm) of a conjugation reaction between intermediate serogroup W-135 polysaccharide and tetanus toxoid as discussed in Example 3.

### DETAILED DESCRIPTION OF THE EMBODIMENTS

Reference will now be made in detail to certain embodiments of the invention, examples of which are illustrated in the accompanying drawings. While embodiments of the invention will be described, it will be understood that they are not intended to limit the invention to those embodiments. On the contrary, the invention is intended to cover all alternatives, modifications, and equivalents of the embodiments described herein.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Similarly, the phrase “at least one” includes should be understood to include one and a plurality. Thus, for example, the phrases “a conjugate” and “at least one conjugate” should both be understood to include one conjugate and a plurality of conjugates.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### A. Definitions

Unless stated otherwise, the following terms and phrases as used herein are intended to have the following meanings:

The terms “percent” or “%” as it relates to the amount of sodium borohydride in a sodium cyanoborohydride reagent refers to percent by weight. For example, 5 μg sodium borohydride in a 5 mg sample of a sodium cyanoborohydride reagent would be 0.5%.

The word “or” is used in the inclusive sense, i.e., equivalent to “and/or,” unless the context requires otherwise.

The terms “linker” and “linkage” are used interchangeably and mean a chemical moiety comprising a chain of atoms that covalently attaches, or is attached to, items such as a carrier protein or a saccharide.

“Linking moiety” means a chemically reactive group, substituent or moiety, e.g. a nucleophile or electrophile, capable of reacting with another molecule to form a linkage by a covalent bond.

“Spacer” is used herein to mean a linker that is made of carbon atoms. For example, a spacer may comprise 2-10 linear carbon atoms.

The term “saccharide” includes both monosaccharides and polysaccharides.

The term “polysaccharide” refers to a chain of covalently linked monosaccharide units, which may be linear or branched. In some embodiments, a polysaccharide has a molecular weight (e.g., weight-average molecular weight or number-average molecular weight) in the range of 300 kDa to 1500 kDa.

The term “about” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### B. Exemplary Methods and Uses of Conjugating at Least One Activated Saccharide to at Least One Protein Carrier and Exemplary Conjugates Produced According to Those Methods and Uses

In some embodiments, a method for conjugating at least one activated saccharide to at least one protein carrier is provided. In some embodiments, the method comprises reacting at least one activated saccharide with the at least one protein carrier. In some embodiments, the method comprises reacting at least one activated saccharide with the at least one protein carrier in the presence of a sodium cyanoborohydride reagent containing no more than about 0.7% of sodium borohydride to obtain a conjugate. Here and throughout, percentage concentrations are w/w unless indicated otherwise.

In some embodiments, a use of a sodium cyanoborohydride reagent as a reagent in conjugating at least one activated saccharide to at least one protein carrier by reductive amination is provided. In some embodiments, the use comprises using a sodium cyanoborohydride reagent containing no more than about 0.7% of sodium borohydride as a reagent in conjugating at least one activated saccharide to at least one protein carrier by reductive amination.

General features of procedures using sodium cyanoborohydride to conjugate a saccharide with a protein carrier are available, e.g., in U.S. Pat. Nos. 4,365,170; 4,673,574; EP 0161188; EP 0208375; EP 0477508; and WO 2018/045286. These documents do not discuss limitation of the amount of sodium borohydride in the sodium cyanoborohydride reagent.

In some embodiments, the methods or uses disclosed herein comprise direct reductive amination.

In some embodiments, in the methods and uses disclosed herein, the at least one activated saccharide is conjugated directly to the at least one carrier protein. In some embodiments, the at least one activated saccharide is conjugated indirectly via a linker group to an amino group on the at least one carrier protein. In some embodiments, the linker group comprises a carbonyl, a carbamate, a spacer, an amide, or a hydrazide linker. In some embodiments, the linker group comprises a dihydrazide linker.

In some embodiments, the methods and uses disclosed herein further comprise activating at least one saccharide with an activating agent to obtain the at least one activated saccharide. Different activation strategies can be applied to the at least one polysaccharide, depending on its structure, in order to facilitate covalently coupling it to a carrier protein. See, e.g., Vaccine Analysis: Strategies, Principles, and Control; Nunnally, B. K., Turula, V. E., Sitrin, R. D., Eds.; Springer: Berlin/Heidelberg, Germany, 2015; pp. 313-320.

Methods for activating saccharides can be accomplished by procedures known in the art—see, e.g., WO 2018/045286 which describes an exemplary activation procedure. A general review of various activation strategies can be found in Vaccine Analysis: Strategies, Principles, and Control; Nunnally, B. K., Turula, V. E., Sitrin, R. D., Eds.; Springer: Berlin/Heidelberg, Germany, 2015; pp. 313-320.

In some embodiments, activation is accomplished by reacting the saccharide with sodium periodate, 1-ethyl-3-(-3dimethylaminopropyl) carbodiimide hydrochloride (EDAC), 1-cyano-4-dimethylaminopyridinium tetrafluoroborate (CDAP) or carbonyl diimidazole (CDI). In some embodiments, the saccharide is activated to introduce aldehyde moieties to provide an activated saccharide. In some embodiments, the introduction of aldehyde moieties is accomplished by reacting the saccharide with sodium periodate.

In some embodiments, an activated saccharide is a saccharide that comprises a moiety suitable for conjugation to a protein carrier in a reaction, e.g., in the presence of a cyanoborohydride reagent, such as a reductive amination reaction. In some embodiments, the moiety suitable for conjugation to a protein carrier in a reaction comprises an ester, aldehyde, —OCN, —CN, —OC(O)NH(CH₂)₃NH₂, —NH₂, —NHNHC(O)(CH₂)ₙC(O)NHNH₂, —NHC(O)(CH₂)ₙC(O)O-pyrrolidine-2,5-dionyl, —NHC(O)CH₂SH, and —NHC(O)(CH₂)ₙC≡CH wherein n in each instance is the number of CH₂ units in the chain.

In some embodiments, the sodium cyanoborohydride reagent in the methods and uses disclosed herein contains no more than about 0.6% of sodium borohydride. In some embodiments, the sodium cyanoborohydride reagent contains no more than about 0.5% of sodium borohydride.

Any known saccharide can be used in the methods and uses disclosed herein—see, e.g., WO 2018/045286; WO 2008/135514; U.S. Pat. No. 7,862,823. In some embodiments, in the methods and uses disclosed herein, the at least one activated saccharide comprises an activated form of a surface carbohydrate of a cell or virus. Exemplary cell types include bacteria, protozoa, helminths, fungi and cancer cells. See, e.g., Astronomo et al., “Carbohydrate vaccines: developing sweet solutions to sticky solutions?” Nat. Rev. Drug Discov., 2010 April 9(4): 10.1038/nrd3012.

In some embodiments, in the methods and uses disclosed herein, the at least one activated saccharide comprises an activated form of a bacterial capsular polysaccharide. Capsular polysaccharides may be prepared according to known methods. See, e.g., U.S. Patent Application Publication No. 2003/0068336 at Example 1; U.S. Pat. No. 6,933,137.

In some embodiments, the at least one activated saccharide comprises an activated form of a bacterial capsular polysaccharide from Haemophilus influenzae. In some embodiments, the Haemophilus influenzae is Haemophilus influenzae type b. In some embodiments, the at least one activated saccharide comprises an activated form of a bacterial capsular polysaccharide from Streptococcus pneumoniae. In some embodiments, the at least one activated saccharide comprises an activated form of a bacterial capsular polysaccharide from Neisseria meningitidis. In some embodiments, the Neisseria meningitidis is serogroup C, A, W-135 or Y.

Any known protein carrier can be used in the methods and uses disclosed herein. Examples of protein carriers are discussed in, e.g., Pichichero M E. “Protein carriers of conjugate vaccines: Characteristics, development, and clinical trials.” Human Vaccines & Immunotherapeutics, 2013; 9(12):2505-2523. Doi: 10.4161/hv.26109, which is incorporated herein by reference. In some embodiments, in the methods and uses disclosed herein, the at least one protein carrier comprises recombinant exoprotein alpha (REP A), Outer Membrane Protein Complex (OMPC), diphtheria toxoid, CRM197, tetanus toxoid or tetanus toxin C fragment. In some embodiments, the at least one protein carrier comprises diphtheria toxoid, CRM197 or tetanus toxoid. In some embodiments, the at least one protein carrier comprises tetanus toxoid. In some embodiments, the tetanus toxoid is a genetically detoxified tetanus toxoid.

In some embodiments, the tetanus toxoid (TT) is prepared by extraction, ammonium sulfate purification, and formalin inactivation of the toxin from cultures of Clostridium tetani (Harvard Strain) grown in a Mueller and Miller medium or a modified Mueller and Miller medium. In some embodiments, the TT is processed to reduce residual formaldehyde, is concentrated in sodium chloride and is filter sterilized. In some embodiments, the TT is purified by chromatography rather than ammonium sulfate purification. In some embodiments, the modified Mueller and Miller medium does not contain beef heart infusion. In some embodiments, the Clostridium tetani is grown in the medium described in WO2006/042542 at Table 3, page 16.

In some embodiments, in the methods and uses disclosed herein, the pH of the reaction is in a range from about 7 to about 10. In some embodiments, the pH is about 7, 8, 9, or 10. In some embodiments, the pH is about 7, 7.5, 8, 8.5, 9, 9.5 or 10. In some embodiments, the pH is about 7, 7.25, 7.5, 7.75, 8, 8.25, 8.50, 8.75, 9, 9.25, 9.5, 9.75 or 10. In some embodiments, the pH is about 8. In some embodiments, the pH is about 9.

In some embodiments, the methods and uses disclosed herein further comprise determining the amount of sodium borohydride in the sodium cyanoborohydride reagent. In some embodiments, the quantitating comprises performing NMR on the sodium cyanoborohydride reagent. In some embodiments, the NMR comprises 1-dimensional proton NMR.

In some embodiments, a conjugate of at least one activated saccharide with the at least one protein carrier is provided. In some embodiments, the conjugates are produced according to the methods and uses disclosed herein.

In some embodiments, the conjugate may be a Haemophilus influenzae polysaccharide conjugated to recombinant exoprotein alpha (REP A), Outer Membrane Protein Complex (OMPC), diphtheria toxoid, CRM197, tetanus toxoid or tetanus toxin C fragment. In some embodiments, the conjugate may be a Streptococcus pneumoniae polysaccharide conjugated to recombinant exoprotein alpha (REP A), Outer Membrane Protein Complex (OMPC), diphtheria toxoid, CRM197, tetanus toxoid or tetanus toxin C fragment. In some embodiments, the conjugate may be Neisseria meningitidis polysaccharide conjugated to recombinant exoprotein alpha (REP A), Outer Membrane Protein Complex (OMPC), diphtheria toxoid, CRM197, tetanus toxoid or tetanus toxin C fragment. In some embodiments, the conjugate is a Neisseria meningitidis polysaccharide tetanus toxoid conjugate.

### C. Exemplary Methods for Quantifying the Amount of Sodium Borohydride in a Sodium Cyanoborohydride Reagent

In some embodiments, a method for quantitating the amount of sodium borohydride in a sodium cyanoborohydride reagent is provided. In some embodiments, the method comprises subjecting a sample of a sodium cyanoborohydride reagent to 1-dimensional proton NMR, thereby obtaining NMR data. In some embodiments, the method comprises determining the amount of sodium borohydride in the sample from the NMR data.

In some embodiments, in the methods disclosed herein, the NMR is carried out in a deuterated solvent. In some embodiments, the deuterated solvent comprises the deuterated form(s) of at least one of dimethyl sulfoxide, chloroform, or methylene chloride. In some embodiments, the NMR is carried out in deuterated dimethyl sulfoxide.

In some embodiments, in the methods disclosed herein, the NMR comprises at least one scan with a relaxation delay at about 41 to 50 seconds. In some embodiments, the NMR comprises at least one scan with a relaxation delay at about 43 to 48 seconds. In some embodiments, the NMR comprises at least one scan with a relaxation delay at about 45 seconds.

In some embodiments, in the methods disclosed herein, the NMR is carried out at a temperature ranging from about 20° C. to about 45° C. In some embodiments, the NMR is carried out at a temperature ranging from about 28° C. to about 40° C. In some embodiments, the NMR is carried out at a temperature ranging from about 30° C. to about 35° C. In some embodiments, the NMR is carried out at a temperature of about 30° C.

In some embodiments, in the methods disclosed herein, the NMR is carried out with the parameters: temperature—about 30° C.; pulsewidth—pw90; spectral width—7000 Hz; and relaxation delay—about 45 second.

In some embodiments, in the methods disclosed herein, the determining step comprises using the peak area at about 0.57 ppm for sodium borohydride resonance as referenced from a solvent resonance. In some embodiments, in the methods disclosed herein, the solvent resonance comprises a dimethyl sulfoxide resonance at about 2.5 ppm.

In some embodiments, in the methods disclosed herein, the determining step uses an external standard curve. In some embodiments, the external standard is of sodium borohydride from about 20-120 μg/ml. In some embodiments, the external standard curve comprises a 6-point external standard curve.

### D. Exemplary Vaccine Compositions and Related Methods and Uses

In some embodiments, a vaccine composition is provided. In some embodiments, the vaccine composition comprising at least one protein conjugated saccharide. In some embodiments, the at least one protein conjugated saccharide is produced according to the methods and uses disclosed herein.

Formulation of the vaccine compositions disclosed herein can be accomplished using art recognized methods. The choice of suitable carriers and other additives will depend on the exact route of administration and the nature of the particular dosage form.

In some embodiments, the at least one protein conjugated saccharide is purified. One method for purification, involving ultrafiltration in the presence of ammonium sulfate, is described in U.S. Pat. No. 6,146,902. Alternatively, conjugates can be purified away from unreacted protein and polysaccharide by any number of standard techniques including, inter alia, size exclusion chromatography, density gradient centrifugation, hydrophobic interaction chromatography, mixed mode resin chromatography, or ammonium sulfate fractionation. See, e.g., P. W. Anderson, et. Al. (1986). J. Immunol. 137: 1181-1186; see also H. J. Jennings and C. Lugowski (1981) J. Immunol. 127: 1011-1018.

In some embodiments, the vaccine compositions disclosed herein further comprise a pharmaceutically acceptable preservative, carrier, buffer excipient, or the like. In one embodiment, the pharmaceutically acceptable preservative, carrier, or excipient increases or extends the shelf life of the compositions.

In some embodiments, the vaccine composition disclosed herein comprises a buffer, such as a pharmaceutically acceptable buffer. In some embodiments, the pharmaceutically acceptable buffer comprises a phosphate buffer, acetate buffer, borate buffer, histidine buffer or succinate buffer. In some embodiments, the pharmaceutically acceptable buffer comprises sodium phosphate. In some embodiments, the pharmaceutically acceptable buffer comprises sodium acetate. In some embodiments, the buffer is present at a concentration ranging from 10 mM to 100 mM, for example, 10 mM to 70 mM, 15 mM to 45 mM, 20 mM to 40 mM, 40 mM to 60 mM, or 60 mM to 100 mM. In some embodiments, buffer is present at 30 mM. In some embodiments, the buffer has a pH of 4.5 to 7.5, 4.5 to 7.0, 4.5 to 6.5, 4.5 to 6.0, 4.5 to 5.5, or 4.5 to 5.0. In some embodiments, the buffer has a pH ranging from 5.5 to 7.0, for example, 5.75 to 6.25, or 6.25 to 6.75. In some embodiments, the buffer has a pH of 5.5 to 6.5. In some embodiments, the buffer has a pH of 5 or 6.

In some embodiments, the vaccine composition disclosed herein further comprise a pharmaceutically acceptable salt. In some embodiments, the vaccine composition disclosed herein comprises saline. In some embodiments, the saline comprises or is NaCl. The NaCl may be present at a concentration of 0.45% to 0.9% w/v, such as 0.5% to 0.85% w/v, or 0.6% to 0.8% w/v, or 0.6%, 0.67%, 0.75%, 0.8%, 0.85%, or 0.9%. In some embodiments, the NaCl may be present at a concentration of about 0.67%.

In some embodiments, the vaccine compositions disclosed herein contain one or more adjuvants. Adjuvants include, by way of example and not limitation, aluminum adjuvants, Freund's Adjuvant, BAY, DC-chol, pcpp, monophoshoryl lipid A, CpG, QS-21, cholera toxin and formyl methionyl peptide. See, e.g., Vaccine Design, the Subunit and Adjuvant Approach, 1995 (M. F. Powell and M. J. Newman, eds., Plenum Press, N.Y.). The adjuvant, if present, can be an aluminum adjuvant, such as aluminum hydroxide or aluminum phosphate. In some embodiments, the vaccine compositions disclosed herein does not comprise adjuvant. In some embodiments, the vaccine compositions disclosed herein comprises adjuvant.

In some embodiments, a vaccine composition disclosed herein is formulated for intramuscular administration. In some embodiments, the administration is subcutaneous, intradermal, intraperitoneal, parenteral or intravenous. Compositions may be in admixture with a suitable carrier, diluent, or excipient such as a sodium acetate buffered saline solution, sterile water, physiological saline or the like. The compositions disclosed herein can also be lyophilized. The compositions disclosed herein can contain auxiliary substances such as wetting or emulsifying agents, pH buffering agents, gelling or viscosity enhancing additives, preservatives, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

In some embodiments, a vaccine composition disclosed herein is provided as a liquid formulation. In some embodiments, the liquid formulation is provided in a syringe, e.g., a pre-filled and/or silicone-free syringe. In some embodiments, such a syringe is commercially packaged for sale and/or distribution.

In some embodiments, the vaccine compositions disclosed herein can be administered as a single dose or in a series (i.e., with a “booster” or “boosters”), or as a booster after earlier administration of a different vaccine, such as a Neisseria meningitidis capsular saccharide conjugate vaccine. For example, a child could receive a single dose early in life, then be administered a booster dose up to ten years later, as is currently recommended for other vaccines to prevent childhood diseases. In some embodiments, a dose of a vaccine disclosed herein is administered two months to ten years after a previously administered vaccine, such as two to four months, four to six months, six to twelve months, 1 year to 2 years, 2 years to 3 years, 3 years to 4 years, 4 years to 5 years, 5 years to 6 years, 6 years to 7 years, 7 years to 8 years, 8 years to 9 years, or 9 years to 10 years after the previously administered vaccine.

In some embodiments, the vaccine compositions disclosed herein are formulated as a single unit dose. In some embodiments, the single unit dose comprises from about 2 μg to about 15 μg of the saccharide. In some embodiments, the single unit dose comprises about 2, 3, 4, 5, 6, 7, 8, 9, 10, 11, 12, 13, 14, or 15 μg of the saccharide. In some embodiments, the single unit dose comprises about 2 μg of the saccharide. In some embodiments, the single unit dose comprises about 4 μg of the saccharide. In some embodiments, the single unit dose comprises about 10 μg of the saccharide. In some embodiments, wherein the vaccine is a multivalent vaccine, the single unit dose comprises from about 2 to about 15 μg each of a plurality of saccharides.

In some embodiments, the carrier protein is present in an amount from about 15 μg to about 80 μg in the single unit dose. In some embodiments, the carrier protein is present in an amount of about 15, 20, 30, 40, 50, 60, 70, or 80 μg in the single unit dose. In some embodiments, the carrier protein is present in an amount of about 15 μg to about 30 μg in a single unit dose. In some embodiments, the carrier protein is present in an amount of about 30 μg to about 45 μg in a single unit dose. In some embodiments, the carrier protein is present in an amount of about 45 μg to about 65 μg in a single unit dose. In some embodiments, the carrier protein is present in an amount of about 25 μg in the single unit dose. In some embodiments, the carrier protein is present in an amount of about 55 μg in the single unit dose.

In some embodiments, a method of vaccinating a subject is provided. In some embodiments, the method comprises administering a dose of the vaccine composition disclosed herein. In some embodiments, a use of the vaccine composition of to immunize a subject is provided. In some embodiments, the use comprises administering a dose of the vaccine composition disclosed herein. In some embodiments, a use of the vaccine composition of for the manufacture of a medicament for immunizing a subject. In some embodiments, the use comprises administering a vaccine composition disclosed herein.

In some embodiments, in the methods or uses disclosed herein, the subject is being immunized against Haemophilus influenzae. In some embodiments, the Haemophilus influenzae is Haemophilus influenzae type b. In some embodiments, the subject is being immunized against Neisseria meningitidis. In some embodiments, the subject is being immunized against Streptococcus pneumoniae.

In some embodiments, in the methods or uses disclosed herein, the vaccine composition is administered intramuscularly. In some embodiments, the administration is subcutaneous, intradermal, intraperitoneal, parenteral or intravenous. Compositions may be in admixture with a suitable carrier, diluent, or excipient such as a sodium acetate buffered saline solution, sterile water, physiological saline or the like. The compositions disclosed herein can also be lyophilized. The compositions disclosed herein can contain auxiliary substances such as wetting or emulsifying agents, pH buffering agents, gelling or viscosity enhancing additives, preservatives, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

In some embodiments, a method of preparing a vaccine composition is disclosed herein. In some embodiments, the method comprises conjugating at least one activated saccharide to at least one protein carrier. In some embodiments, the conjugating at least one activated saccharide to at least one protein carrier is carrier out according to the methods or uses disclosed herein. In some embodiments, the method further comprises formulating the conjugate into a vaccine composition.

In some embodiments, a method of preparing at least one vaccine composition comprising a conjugate is provided. In some embodiments, the method comprising the steps of: determining the amount of sodium borohydride in a sodium cyanoborohydride reagent; selecting a sodium cyanoborohydride reagent containing no more than about 0.7% of sodium borohydride; reacting at least one activated saccharide with at least one protein carrier in presence of the selected sodium cyanoborohydride reagent, thereby providing at least one conjugate; and formulating the at least one conjugate into a vaccine composition.

In some embodiments, the methods disclosed herein further comprises activating at least one saccharide with an activating agent to obtain the at least one activated saccharide. As discussed above, methods of activation are known in the art.

In some embodiments, in the methods disclosed herein, the selected sodium cyanoborohydride reagent contains no more than about 0.6% of sodium borohydride. In some embodiments, the selected sodium cyanoborohydride reagent contains no more than about 0.5% of sodium borohydride.

In some embodiments, in the methods disclosed herein, the at least one activated saccharide comprises an activated form of a surface carbohydrate of a cell or virus. In some embodiments, the at least one activated saccharide comprises an activated form of a bacterial capsular polysaccharide. In some embodiments, the at least one activated saccharide comprises an activated form of a bacterial capsular polysaccharide from Haemophilus influenzae. In some embodiments, the Haemophilus influenzae is Haemophilus influenzae type b. In some embodiments, the at least one activated saccharide comprises an activated form of a bacterial capsular polysaccharide from Streptococcus pneumoniae. In some embodiments, the at least one activated saccharide comprises an activated form of a bacterial capsular polysaccharide from Neisseria meningitidis. In some embodiments, the Neisseria meningitidis is serogroup C, A, W-135 or Y.

In some embodiments, in the methods disclosed herein, the at least one protein carrier comprises recombinant exoprotein alpha (REP A), Outer Membrane Protein Complex (OMPC), diphtheria toxoid, CRM197, tetanus toxoid or tetanus toxin C fragment. In some embodiments, the at least one protein carrier comprises diphtheria toxoid, CRM197 or tetanus toxoid. In some embodiments, the at least one protein carrier comprises tetanus toxoid. In some embodiments, the at least one protein carrier comprises a genetically detoxified tetanus toxoid.

### EXAMPLES

The following are examples of methods, uses, conjugates, and compositions disclosed herein. It is understood that various other embodiments may be practiced, given the general and detailed descriptions provided above. The following examples are given for the purpose of illustrating the present teachings and shall not be construed as being a limitation on the scope of the disclosure or claims.

Abbreviations used:

| | |
|---|---|
| ¹H NMR | Proton NMR |
| 1-D Proton NMR or | 1-Dimensional Proton NMR |
| 1-D ¹H NMR | |
| ¹¹Br NMR | Boron NMR |
| DMSO-d6 | Dimethyl sulfoxide-d6 |
| HPSEC | High Performance Size Exclusion Chromatography |
| SEC | Size Exclusion Chromatography |
| BCA | Bicinchoninic Acid |

### Example 1. Development of a Method for Quantitative 1-Dimensional Proton NMR to Quantitate Sodium Borohydride in the Raw Material Sodium Cyanoborohydride

The amount of NaBH₄ in a NaCNBH₄ reagent was conducted using 1-D proton NMR. The method used an external 6-point standard curve of sodium borohydride from about 20-120 μg/mL with a quantitation range of approximately 30-300 μg/mL with a quantitation limit of approximately 30 μg/mL.

Quantitation was accomplished with linear regression using the peak area sodium borohydride peak at a chemical shift value of approximately-0.57 ppm as referenced from the dimethyl sulfoxide resonance at 2.5 ppm.

### Initial Method Development

To evaluate method feasibility, a sample consisting of 50 mg/mL sodium cyanoborohydride and 0.050 mg/mL sodium borohydride was prepared in d-DMSO. NMR analysis was performed using the instrument parameters in Table 1, below.

TABLE 1
Acquisition Parameters

| Parameter | Value | |
|---|---|---|
| Temperature (temp) | 30° | C. |
| Receiver Gain (gain) | 36 | |
| Pulsewidth (sw) | pw90 | |
| Spectral Width (sw) | 7000 | Hz |

| | |
|---|---|
| Number of points (np) | 64K |
| Number of scans/transients (nt) | 32 |
| Steady state pulses (ss) | 4 |
| Relaxation delay (d1) | 45 |

### Proton NMR of Borohydride Compounds

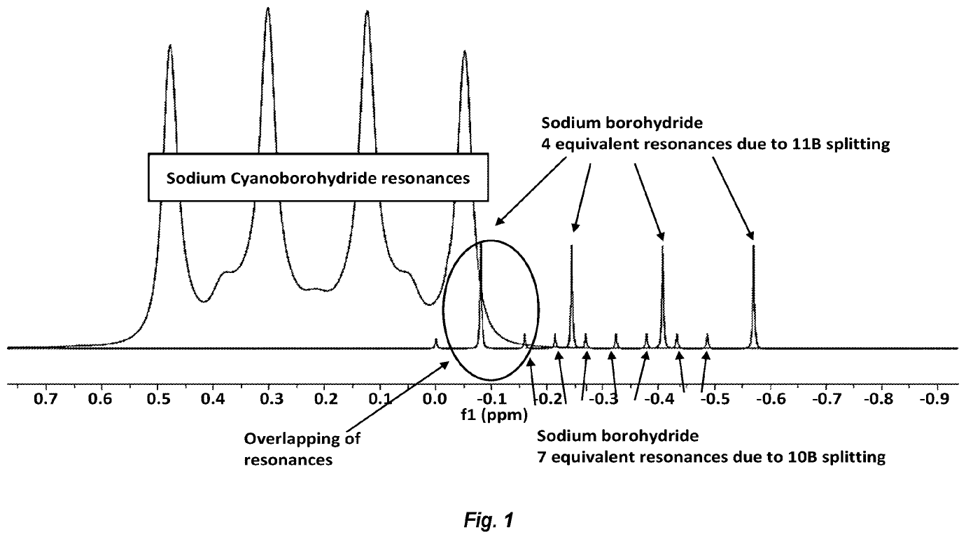

The resonances in the proton spectrum for sodium borohydride and sodium cyanoborohydride exhibit splitting patterns due to the bonding with boron. Boron has two magnetically active isotopes, ¹¹B and ¹⁰B, the properties of which are shown in the Table 2 below. In the proton spectrum, the signal was split by the coupling to the Boron nuclei. Due to the higher natural abundance of ¹¹B and the spin=3/2, four equally intense single peaks were observed. See FIG. 1. Conversely, the lower natural abundance of ¹⁰B and spin=3 results in seven line multiplet, at a much lower intensity. See id.

The splitting pattern is similar in both sodium borohydride and sodium cyanoborohydride; however, the chemical shift and linewidths of sodium cyanoborohydride were different due to the presence of the cyano-group. See FIG. 1.

TABLE 2
NMR Properties of Boron

| | | | 1H splitting | 1H splitting |
|---|---|---|---|---|
| | Natural | | pattern for | pattern for |
| Isotope | abundance | Spin | NaBH4, n = 1 | NaCNBH3, n = 1 |
| ¹¹B | 80.4% | I = 3/2 | 2nI + 1 = 4 | 2nI + 1 = 4 |
| ¹⁰B | 19.6% | I = 3 | 2nI + 1 = 7 | 2nI + 1 = 7 |

### Temperature Study

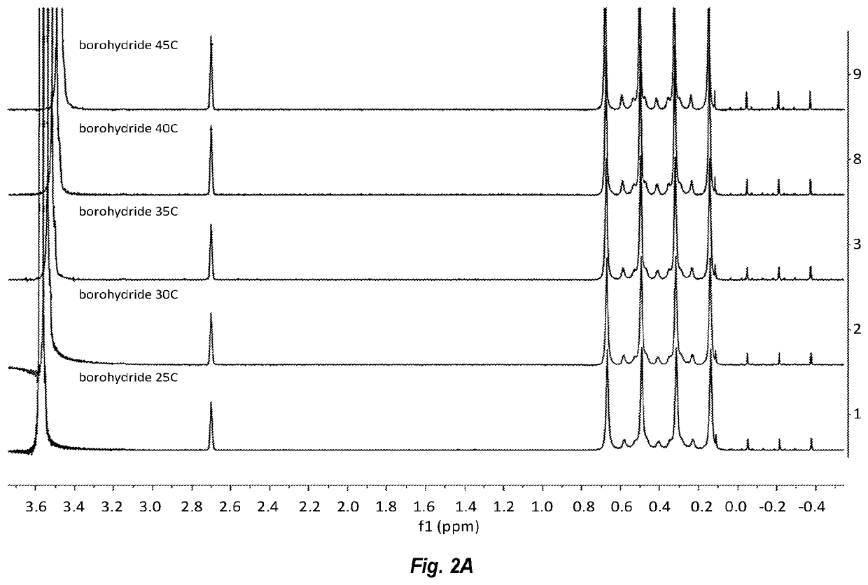

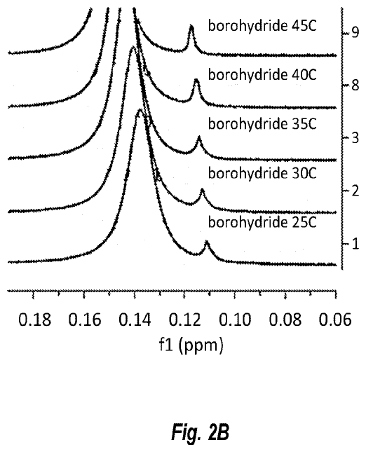

For accurate integration, there needs to be baseline separation of peaks of interest. In particular, if an internal standard is employed, overlapping of the sodium borohydride and the sodium cyanoborohydride resonances introduces a significant source of error if integration is done on the entire resonance, i.e., all peaks of the analyte. As shown in FIG. 1, there is overlap between the sodium borohydride and sodium cyanoborohydride. By varying the temperature, the resonances for sodium cyanoborohydride exhibited a slight variation in chemical shift values, however, even at elevated temperatures, baseline separation was not achieved. See FIG. 2. Therefore, an acquisition temperature of 30° C. was maintained.

Material without Sodium Borohydride

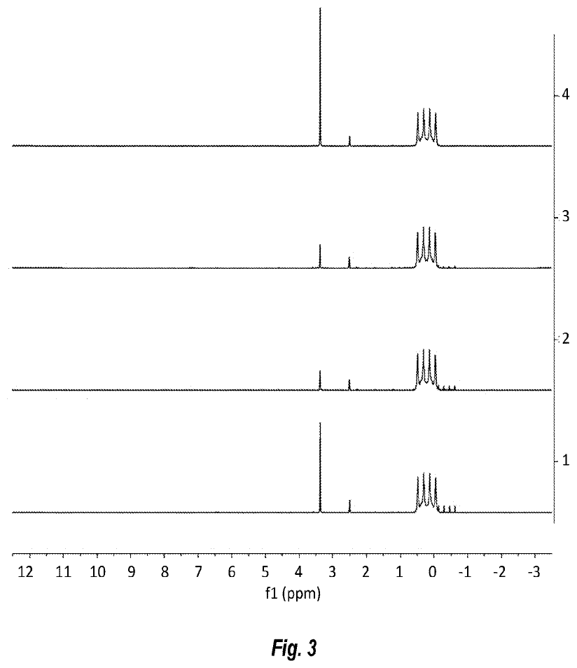

Various sodium cyanoborohydride lots were prepared for NMR analysis in order to find a lot of material that contained no measurable amount of sodium borohydride. See FIG. 3. Because Lot 4 did not show any measurable amounts of sodium borohydride, this material was chosen for method development work.

### Peak for Quantification

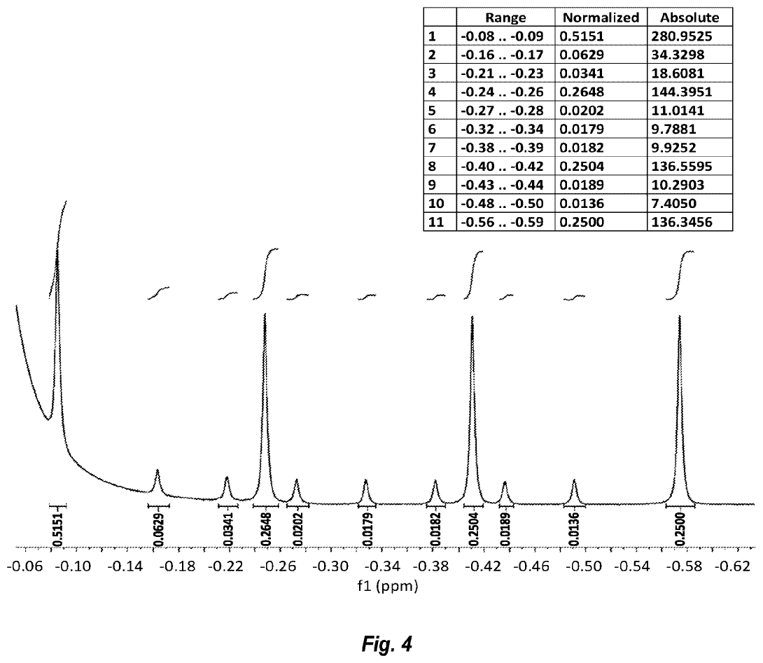

As shown previously in FIG. 1, the sodium cyanoborohydride resonance overlap with part of the sodium borohydride peaks would cause an error in quantitation. FIG. 4 shows integral values for all the sodium borohydride peaks in Lot 4 that was spiked with 50 μg/mL sodium borohydride. The presence of the large sodium cyanoborohydride resonance contributes to distortion of the baseline for the leftmost sodium borohydride resonances. The resonance at −0.57 ppm exhibits a flat baseline and is well resolved from neighboring peaks. Thus, this peak was chosen for quantitation.

### Determination of Spin-Lattice Relaxation Time

An inversion recovery experiment was conducted to determine spin-lattice relaxation times (T₁) for the analyte. T₁ is a measure of the time that each nucleus needs to reach equilibrium with its surroundings in the magnetic field. Accurate estimates of T₁ ensure adequate relaxation delay (d2) between scans to allow magnetization to return to an equilibrium state. For an experiment in which multiple scans are collected (nt>1), if a value of d2 is used that is too short, the magnetization will not be fully restored to an equilibrium value before the start of the next scan resulting in errors in integration.

In the inversion recovery experiment, the magnetization (M₀) is inverted with a 180° pulse then a time τ (d2) is applied to allow for relaxation. The magnetization after time, t, is measured applying a 90° observe pulse. In the experiment, values of τ (or d2) are arrayed.

A 50 mg/ml sodium cyanoborohydride sample from Lot 4 spiked with 0.05 mg/mL sodium borohydride was used for the experiment. The acquisition parameters were 16 scans, a relaxation delay of 45 seconds, 90 pulse width of 8.3 microseconds at a transmitter power of 57 dB, 180 pulse width of 16.6 microseconds, temperature of 30° C., acquisition time of 8.194 s and d2 was arrayed with 15 points for the time between pulses of 0.010 seconds to 60 seconds. The sweep width was 7998.4 Hz with 128K acquired data points.

Peak heights and T₁ analysis were done using the Varian software T₁ analysis using the following equation: M=M₀(1−2e^{-τ/T}₁).

The relaxation delay (d2), i.e., the time delay between pulsed scans in an experiment, is at least five times the longest T₁. With a value of T₁ for the sodium borohydride resonances of approximately 8 s (i.e., 5*T₁=about 40 seconds), a 45 second value for d1 is an adequate relaxation delay time between pulsed scans.

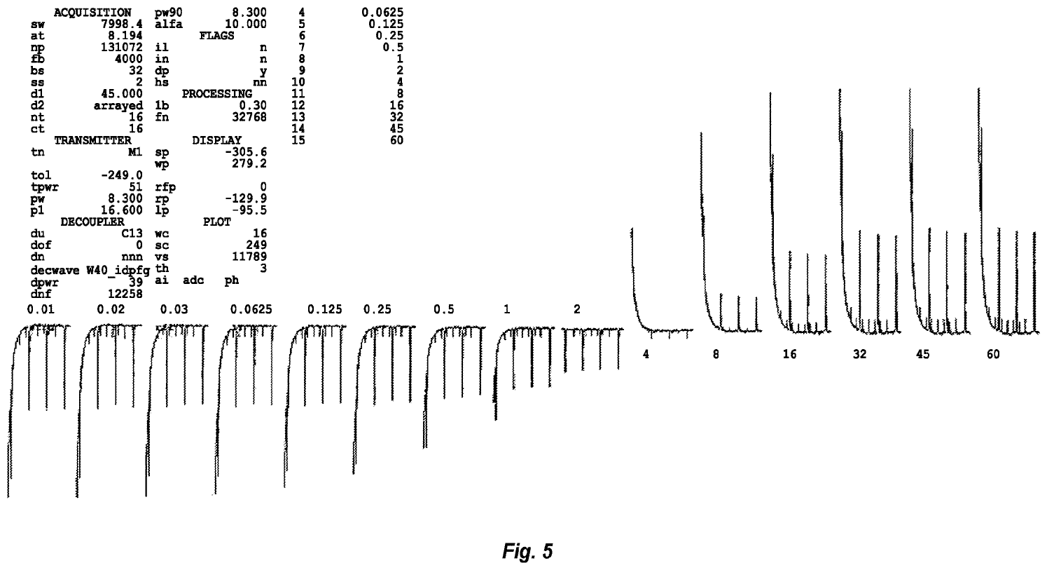

The arrayed spectrum from the T₁ experiment is shown in FIG. 5 and the results of the analysis are shown in Table 3 below.

TABLE 3
Sodium Borohydride Spin Lattice Relaxation Times

| Peak Chemical Shift | T1 (s) | Error (s) |
|---|---|---|
| −0.077* | 5.38 | 0.123 |
| −0.24 | 7.95 | 0.0758 |
| −0.40 | 8.06 | 0.0947 |
| −0.57 | 7.97 | 0.129 |

*Peak overlap with cyanoborohydride

### Initial Recovery Studies

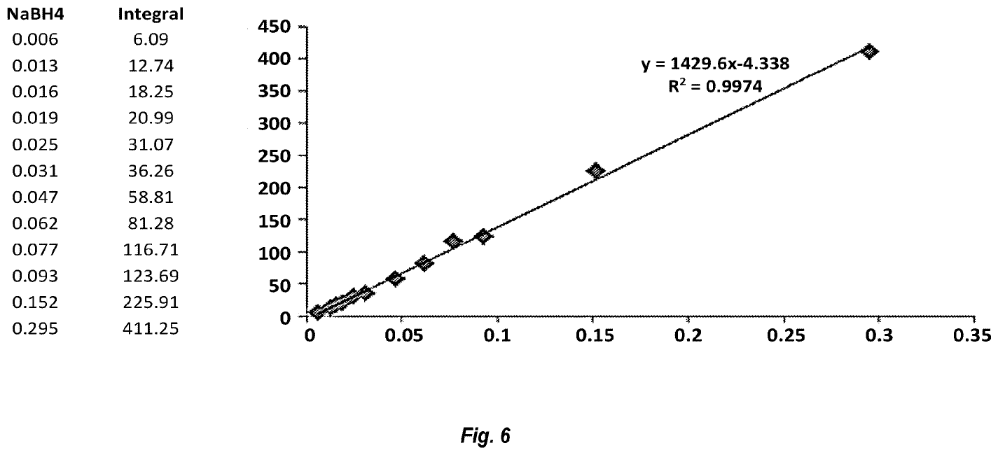

Sodium Borohydride in Dimethyl Sulfoxide-d6—A 4.7 mg/ml stock solution of sodium borohydride in DMSO-d6 was prepared. 750 ml samples were prepared ranging from 6 to 295 ppm. NMR analysis was performed using the following instrument parameters: Temperature 30° C., receiver gain 30, 90° pulsewidth (8.3 μs), spectral width 7998.4 Hz, and 64K acquisition points. After four steady state scans, 32 scans were acquired with a relaxation delay of 45 seconds. A linear curve was generated using the sodium borohydride concentration vs. the peak area of the resonance at −0.57 ppm. See FIG. 6.

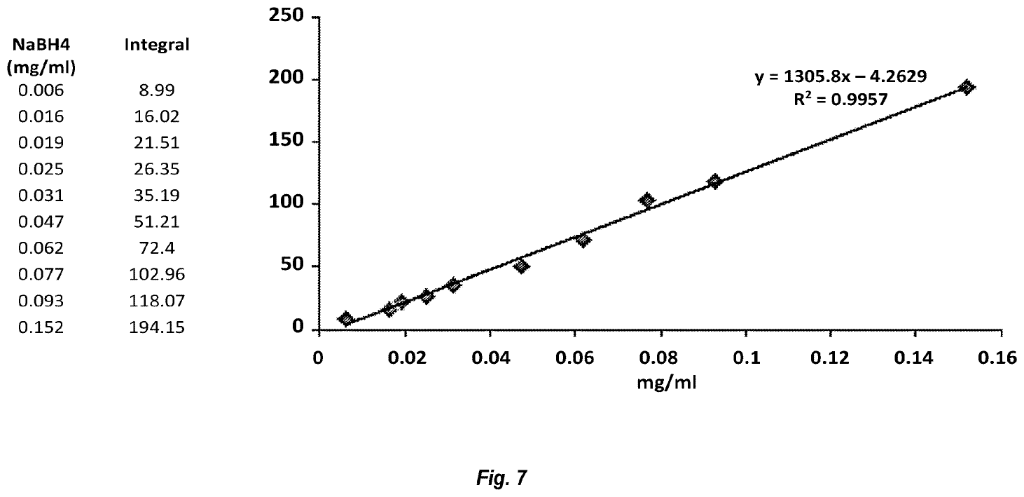

Sodium Borohydride in Sodium Cyanoborohydride Sample Matrix-A 50 mg/mL stock solution of sodium cyanoborohydride was prepared. Using the 4.7 mg/ml sodium borohydride stock, samples at varying concentration of sodium borohydride were prepared for NMR analysis. FIG. 7 shows the data table for the experiment and a plot of concentration vs. peak areas.

### Example 2. Method Assessment

### Accuracy, Precision, and Linearity

A 50 mg/ml solution of sodium cyanoborohydride was prepared. In this sample matrix, 5 levels of sodium borohydride were prepared at 31, 104, 183, 261 and 313 μg/mL. Three samples were analyzed at each level. A six-point external calibration curve was used.

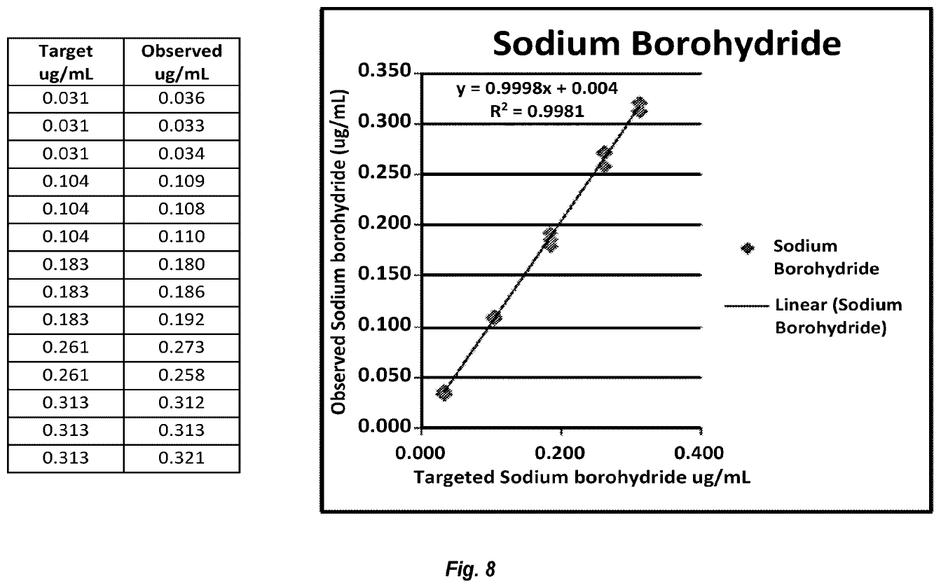

Table 4 below shows the results of the study. Level 1 correspond to 31 μg/mL; Level 2 corresponds to 104 μg/mL; Level 3 corresponds to 183 μg/mL; Level 4 corresponds to 261 μg/mL; and Level 5 corresponds to 313 μg/mL. For accuracy, 80-120% is considered good, with closer to 100% being best. For precision, less than 15% is acceptable but the lower the better. As shown below, the method shows good accuracy and precision (% relative standard deviation). FIG. 8 shows the linearity of the target (i.e., expected borohydride) vs. the observed sodium borohydride in sodium cyanoborohydride.

TABLE 4
Accuracy, Repeatability, and Intermediate Precision

| | Borohydride | Expected | | | Accuracy/ | | |
|---|---|---|---|---|---|---|---|
| | recovered | Borohydride | Accuracy | Average/ | Level | STD | % |
| Sample | (ug/mL) | (ug/mL) | (% recovery) | Level | (% recovery) | DEV | RSD |
| Level 1-A | 36 | 31 | 115 | 0.034 | 110 | 0.0015 | 4.4 |
| Level 1-B | 33 | | 105 | | | | |
| Level 1-C | 34 | | 109 | | | | |
| Level 2-A | 109 | 104 | 104 | 0.109 | 104 | 0.0010 | 0.9 |
| Level 2-B | 108 | | 103 | | | | |
| Level 2-C | 110 | | 105 | | | | |
| Level 3-A | 180 | 183 | 99 | 0.186 | 102 | 0.0060 | 3.2 |
| Level 3-B | 186 | | 102 | | | | |
| Level 3-C | 192 | | 105 | | | | |
| Level 4-A | 273 | 261 | 105 | 0.267 | 102 | 0.0081 | 3.0 |
| Level 4-B | 271 | | 104 | | | | |
| Level 4-C | 258 | | 99 | | | | |
| Level 5-A | 312 | 313 | 100 | 0.315 | 101 | 0.0049 | 1.6 |
| Level 5-B | 313 | | 100 | | | | |
| Level 5-C | 321 | | 103 | | | | |
| | | | Overall % Recovery | | 104 | | |

### Example 3. Threshold at which Sodium Borohydride Contamination Negatively Impacts the Conjugation Reaction

The purpose of this study was to determine the threshold at which sodium borohydride contamination negatively impacts the conjugation reaction.

### Effectiveness of Polysaccharide Activation Prior to Conjugation

A reducing activity test was performed on the activated polysaccharide intermediate (N. meningitidis Polysaccharide Serogroups C, W-135 and Y activated by sodium periodate) to determine the effectiveness of the activation of the polysaccharide. The assay was colorimetric and was based on the formation of purple complex by bicinchoninic acid (BCA) in alkaline solution with Cu⁺ ions obtained by reducing sugar activity on Cu⁺² ions. This test is an indicator used to determine the ability of the activated polysaccharide to conjugate with the tetanus protein in the conjugation reaction. A review of the reducing activity results showed that the polysaccharide was effectively activated and suitable for conjugation.

### Suitability of the Tetanus Toxoid

A review of the protein content for the lot of Tetanus Toxoid used for conjugation was completed. More specifically, the BCA (bicinchoninic acid) Protein Assay was adapted from the BCA colorimetric assay for protein described by Smith, et al., Analytical Biochemistry 1985; 150 (1): 76-85. When combined with the sample protein, Cu⁺² from the BCA working reagent is reduced to Cu⁺¹. Each Cu⁺¹ ion combines with two BCA molecules to form a BCA-Cu⁺¹ complex. This complex produces a purple color that exhibits strong absorbance at 562 nm. The protein concentration was found to be suitable for conjugation.

### Suitability of the Sodium Cyanoborohydride Used for Conjugation

The purpose of this study was to perform an initial screening to determine the threshold at which sodium borohydride contamination negatively affects the conjugation reaction. Four experiments were executed with sodium borohydride concentrations at 0.5%, 1.0%, 1.5% and 2.0%.

Sodium cyanoborohydride was used in the conjugation reaction during the manufacture of N. meningitidis Polysaccharide Tetanus Toxoid Conjugate Concentrate for Serogroups C, W-135 and Y. The pH of the conjugation reaction for serogroup C was 8 and was 9 for serogroups W-135 and Y.

During the conjugation reaction, polysaccharide was added in excess of tetanus to ensure complete utilization of the tetanus. The conjugate concentrate was manufactured by covalent attachment of the depolymerized/activated polysaccharide to the tetanus toxoid carrier protein. This attachment occurred via reductive amination of the aldehyde groups on the polysaccharide chain to the amine groups present on the tetanus toxoid carrier protein. In this reaction, sodium cyanoborohydride specifically reduced Schiff base intermediates formed between the polysaccharide aldehyde groups and the protein amine groups to form stable secondary amine linkages. The resulting conjugate had the polysaccharide chains linked to the protein carrier. The reaction was allowed to mix for 16-24 hours. After the allowed mix time, the conjugate reaction was then capped by adding sodium borohydride. Sodium borohydride reduced the remaining aldehyde groups to hydroxyl groups thereby inhibiting further conjugation. If trace amounts of sodium borohydride were present during the conjugation reaction, the sodium borohydride would have reduced the amount of aldehyde groups available for attachment to the protein carrier, resulting in free protein.

HPSEC analysis was performed to determine differences in the sizes of the conjugate molecule. Larger molecules flow through an SEC column faster than smaller molecules. Therefore, a shift toward a longer retention time indicates a smaller conjugate size which corresponds to the impact on reaction efficiency.

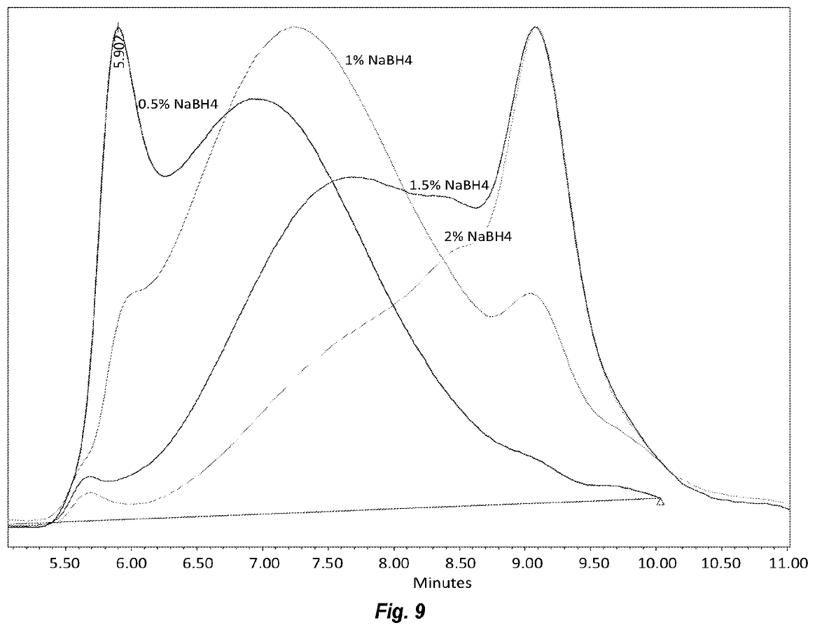

(1) N. meningitidis Polysaccharide Tetanus Toxoid Conjugate Concentrate for Serogroup W: The starting materials used were activated intermediate serogroup W-135 polysaccharide and tetanus toxoid. Pharma grade, 97.5% purity, sodium cyanoborohydride was used for the purpose of this study. Sodium borohydride was then spiked into the sodium cyanoborohydride stock solution at various concentrations and then allowed to mix for 24 hours. After 24 hours, the complete samples were pulled and run on a HPSEC column to determine reaction efficiency. The chromatograms (absorbance 280 nm) are shown in FIG. 9.

The results of this study indicate that concentrations ≥1.0% negatively impact the conjugation reaction—i.e., there is a large amount of free protein left in the reaction.

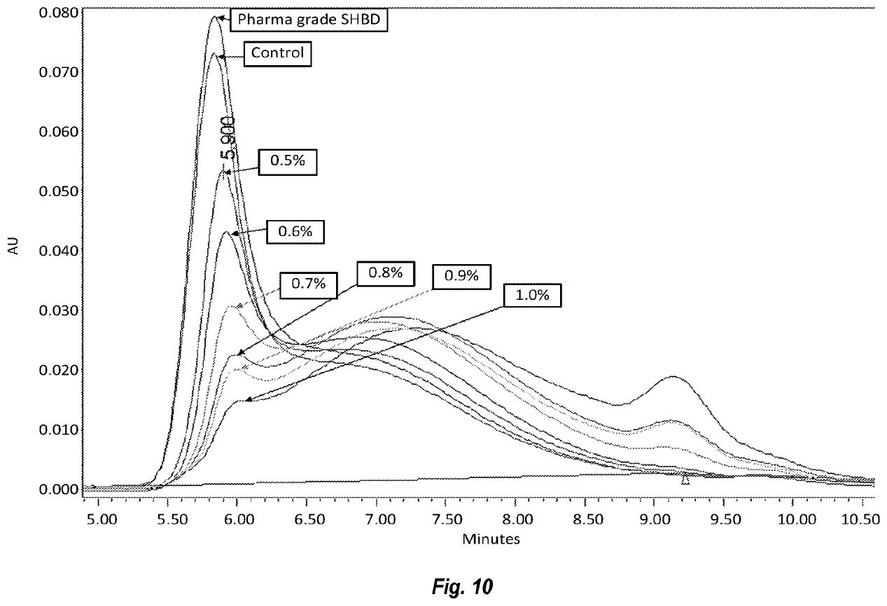

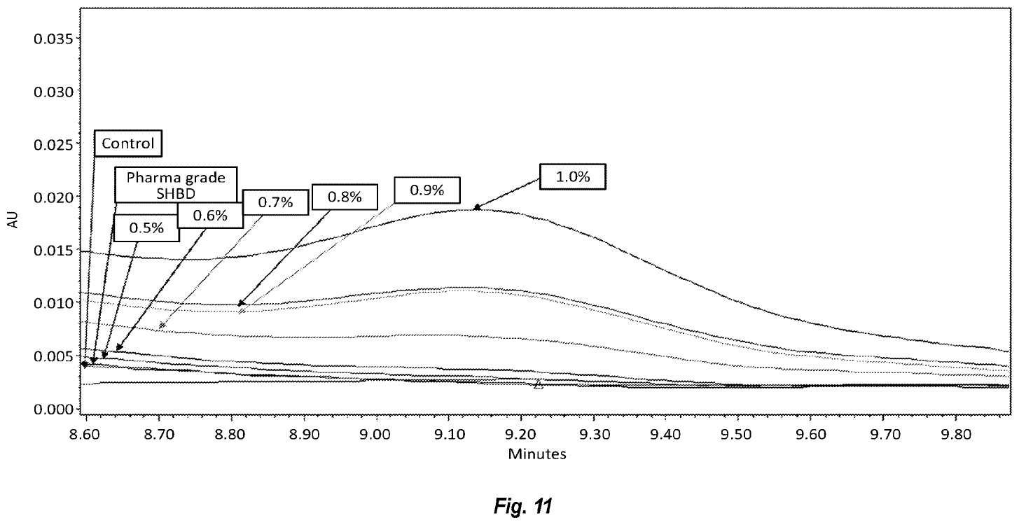

(2) N. meningitidis Polysaccharide Tetanus Toxoid Conjugate Concentrate for Serogroup Y: The starting materials used were activated intermediate serogroup Y polysaccharide and tetanus toxoid. Pharma grade, 97.5% purity, sodium cyanoborohydride was used for the purpose of this study, except for the control arm which used reagent grade sodium cyanoborohydride. Sodium borohydride was then spiked into the sodium cyanoborohydride stock solution at various concentrations (0.5%, 0.6%, 0.7%, 0.8%, 0.9%, 1.0%) and then allowed to mix for 24 hours. After the 24 hours, the complete samples were pulled and run on a size exclusion chromatography column to determine the reaction efficiency. The HPSEC chromatograms (absorbance 280 nm) are shown in FIG. 10 and FIG. 11. The same kind of results is expected for serogroup W polysaccharide (similar structure and same conjugation process). The results of this study indicate that concentrations ≥0.7% negatively impact the conjugation reaction.

### Free Protein Results

A total of 8 runs (see Table 5 below) of N. meningitidis Polysaccharide Tetanus Toxoid Conjugate Concentrate were successfully produced according to the method described in WO2018/045286 to complete evaluation of impact of residual sodium borohydride present in sodium cyanoborohydride.

The experiments consisted of four control arms and four experimental arms that were spiked with sodium borohydride at concentrations of 0.5% and 1.0%. Pharma grade and reagent grade sodium cyanoborohydride was used for the purpose of this study. The appropriate amount of sodium borohydride was spiked into the sodium cyanoborohydride stock solution at the predetermined concentrations prior to being delivered into the conjugation reaction.

TABLE 5
Description of Experimental Arms

| Sero- | | | | |
|---|---|---|---|---|
| group | Run #1 | Run #2 | Run #3 | Run #4 |
| C | Control w/reagent | Control w/pharma | Pharma grade | Pharma grade |
| | grade (95% purity) | grade (97.5% purity) | sodium | sodium |
| | sodium | sodium | cyanoborohydride | cyanoborohydride |
| | cyanoborohydride | cyanoborohydride | spiked w/0.5% | spiked w/1.0% |
| | | | sodium | sodium |
| | | | borohydride | borohydride |
| W | Control w/reagent | Control w/pharma | Pharma grade | Pharma grade |
| | grade (95% purity) | grade (97.5% purity) | sodium | sodium |
| | sodium | sodium | cyanoborohydride | cyanoborohydride |
| | cyanoborohydride | cyanoborohydride | spiked w/0.5% | spiked w/1.0% |
| | | | sodium | sodium |
| | | | borohydride | borohydride |

The material was then processed to completion via diafiltration, hydrophobic interaction chromatography purification and final diafiltration. HPSEC samples were pulled from the crude conjugate mixture after the completion of the 16-24 hour hold time. These samples were pulled to determine if there was a shift in the chromatogram towards a longer retention time. Longer retention times indicate a potential impact on the conjugation reaction.

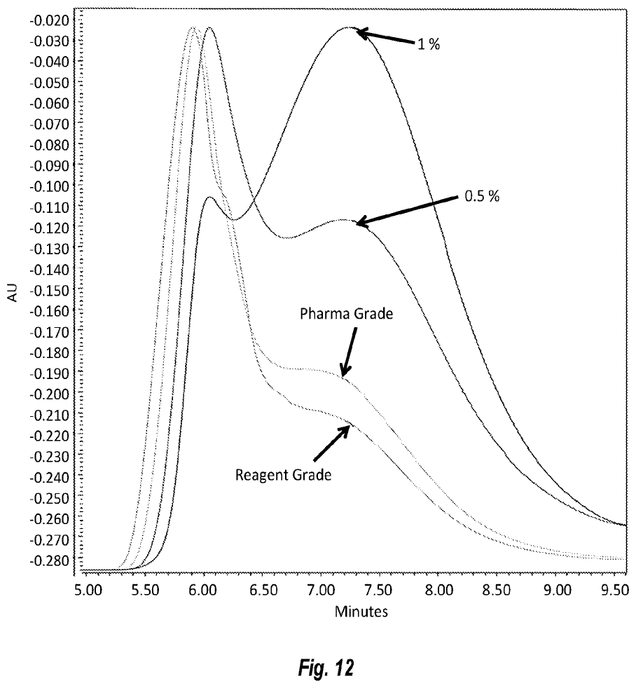

Serogroup C chromatograms (FIG. 12) did not show a significant difference between the pharma grade and reagent grade controls. The serogroup C chromatograms did show a slight shift with the 0.5% sodium borohydride sample and a significant shift with the 1.0% sodium borohydride sample. The significant shift in the 1% chromatogram indicates that 1% sodium borohydride content can negatively impact reaction efficiency.

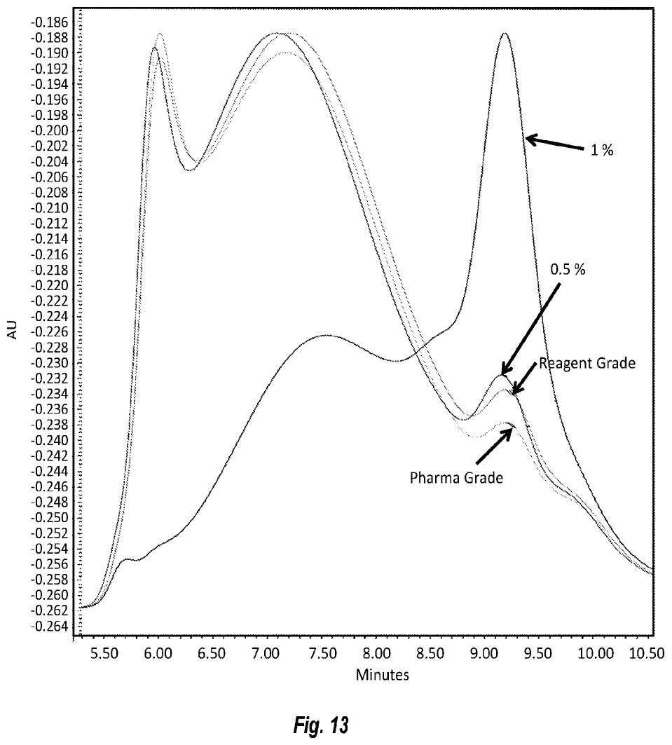

Serogroup W-135 chromatograms (FIG. 13) did not show a significant difference between runs 1, 2 and 3. However the serogroup W-135 (1.0% sodium borohydride) did show a significant shift, indicating that the presence of 1% sodium borohydride content can negatively impact reaction efficiency.

It has been shown that lots of sodium cyanoborohydride containing 0.5% sodium borohydride do not negatively impact the free protein content of the final bulk concentrate. However, lots containing at least 1% sodium borohydride content can cause an increase in free protein content.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1.-64. (canceled)

65. A method for conjugating at least one activated saccharide to at least one protein carrier, the method comprising:
a) determining the amount of sodium borohydride in a sodium cyanoborohydride reagent; and
b) reacting the at least one activated saccharide with the at least one protein carrier in the presence of a sodium cyanoborohydride reagent containing no more than about 0.7% of sodium borohydride to obtain a conjugate.

66. A method for conjugating at least one activated saccharide to at least one protein carrier by reductive amination, the method comprising reacting the at least one activated saccharide with the at least one protein carrier in the presence of a sodium cyanoborohydride reagent, wherein the sodium cyanoborohydride reagent has been determined to contain no more than 0.7% sodium borohydride.

67. The method according to claim 65, wherein the method further comprises activating at least one saccharide with an activating agent to obtain the at least one activated saccharide.

68. The method according to claim 65, wherein the sodium cyanoborohydride reagent contains no more than about 0.6% of sodium borohydride, optionally no more than about 0.5% of sodium borohydride.

69. The method according to claim 65, wherein the at least one activated saccharide comprises an activated form of a surface carbohydrate of a cell or virus.

70. The method according to claim 65, wherein the at least one activated saccharide comprises an activated form of a bacterial capsular polysaccharide, optionally wherein the activated form of a bacterial capsular polysaccharide is selected from Haemophilus influenzae, Streptococcus pneumoniae, and Neisseria meningitidis.

71. The method according to claim 70, wherein the activated saccharide comprises an activated form of a bacterial capsular polysaccharide from Neisseria meningitidis serogroup C, A, W-135 or Y.

72. The method according to claim 65, wherein the at least one protein carrier comprises recombinant exoprotein alpha (REP A), Outer Membrane Protein Complex (OMPC), diphtheria toxoid, CRM197, tetanus toxoid or tetanus toxin C fragment.

73. The method according to claim 65, wherein the pH of the reaction is in a range from about 7 to about 10, optionally wherein the pH is about 8 or about 9.

74. The method according to claim 65, wherein the determining the amount of sodium borohydride in a sodium cyanoborohydride reagent comprises performing NMR on the sodium cyanoborohydride reagent.

75. The method according to claim 74, wherein the NMR comprises 1-dimensional proton NMR,
optionally wherein the NMR is carried out in a deuterated solvent comprising the deuterated form(s) of at least one of dimethyl sulfoxide, chloroform, or methylene chloride,
optionally wherein the NMR comprises at least one scan with a relaxation delay at about 41 to 50 seconds, optionally comprising at least one scan with a relaxation delay at about 43 to 48 seconds, or further optionally at about 45 seconds,
optionally wherein the NMR is carried out at a temperature ranging from about 20° C. to about 45° C., optionally at a temperature ranging from about 28° C. to about 40° C., or ranging from about 30° C. to about 35° C., or further optionally at about 30° C., and/or
optionally wherein the NMR is carried out with the parameters:
temperature: about 30° C.
pulsewidth: pw90
spectral width: 7000 Hz
relaxation delay: about 45 seconds.

76. A method for quantitating the amount of sodium borohydride in a sodium cyanoborohydride reagent, the method comprising:
(a) subjecting a sample of a sodium cyanoborohydride reagent to 1-dimensional proton NMR, thereby obtaining NMR data; and
(b) determining the amount of sodium borohydride in the sample from the NMR data,
optionally wherein the NMR is carried out in a deuterated solvent comprising the deuterated form(s) of at least one of dimethyl sulfoxide, chloroform, or methylene chloride,
optionally wherein the NMR comprises at least one scan with a relaxation delay at about 41 to 50 seconds, optionally comprising at least one scan with a relaxation delay at about 43 to 48 seconds, or further optionally at about 45 seconds,
optionally wherein the NMR is carried out at a temperature ranging from about 20° C. to about 45° C., optionally at a temperature ranging from about 28° C. to about 40° C., or ranging from about 30° C. to about 35° C., or further optionally at about 30° C., and/or
optionally wherein the NMR is carried out with the parameters:
temperature: about 30° C.
pulsewidth: pw90
spectral width: 7000 Hz
relaxation delay: about 45 seconds.

77. The method according to claim 76, wherein determining the amount of sodium borohydride comprises:
using the peak area at about 0.57 ppm for sodium borohydride resonance as referenced from a solvent resonance, optionally wherein the solvent resonance comprises a dimethyl sulfoxide resonance at about 2.5 ppm; and/or
optionally wherein the method further comprises using an external standard curve of sodium borohydride, optionally when the external standard curve of sodium borohydride is from about 20-120 μg/ml, optionally wherein the external standard curve comprises a 6-point external standard curve.

78. A conjugate produced according to the method of claim 65.

79. A method of preparing a vaccine composition, comprising conjugating at least one activated saccharide to at least one protein carrier according to the method of claim 65; and formulating the conjugate into a vaccine composition.

80. The method according to claim 79, wherein the at least one activated saccharide comprises an activated form of a surface carbohydrate of a cell or virus, optionally an activated form of a bacterial capsular polysaccharide, optionally wherein the bacterial capsular polysaccharide is from Haemophilus influenzae, Streptococcus pneumoniae or Neisseria meningitidis, optionally wherein the Neisseria meningitidis is serogroup C, A, W-135 or Y.

81. The method according to claim 79, wherein the at least one protein carrier comprises recombinant exoprotein alpha (REP A), Outer Membrane Protein Complex (OMPC), diphtheria toxoid, CRM197, tetanus toxoid or tetanus toxin C fragment.

82. A method of vaccinating a subject comprising administering to the subject a dose of the vaccine composition produced according to the method according to claim 79.

83. The method according to claim 82, wherein the subject is being immunized against Haemophilus influenzae, Neisseria meningitidis, or Streptococcus pneumoniae.

84. The method according to claim 82, wherein the vaccine composition is administered intramuscularly.

85. A method of preparing at least one vaccine composition comprising a conjugate, the method comprising the steps of:
a) determining the amount of sodium borohydride in a sodium cyanoborohydride reagent,
b) selecting a sodium cyanoborohydride reagent containing no more than about 0.7% of sodium borohydride, optionally containing no more than about 0.6% of sodium borohydride or no more than about 0.5% of sodium borohydride
c) reacting at least one activated saccharide with at least one protein carrier in presence of the selected sodium cyanoborohydride reagent, thereby providing at least one conjugate, and
d) formulating the at least one conjugate into a vaccine composition, optionally wherein the method further comprises activating at least one saccharide with an activating agent to obtain the at least one activated saccharide.

86. A method of preventing Haemophilus influenzae, Neisseria meningitidis or Streptococcus pneumoniae infection or disease, the method comprising administering a dose of a vaccine composition obtained according to claim 82.
